(19) United States
(12) Patent Application Publication
(10) Pub. No.: US 2021/0113669 A1
(43) Pub. Date: Apr. 22, 2021

(54) NEW USE OF CNF1

(71) Applicant: ISTITUTO SUPERIORE DI SANITA, Rome (IT)

(72) Inventors: Carla FIORENTINI, Rome (IT); Francesco SILVESTRINI, Rome (IT); Stefano LOIZZO, Rome (IT); Valeria MESSINA, Rome (IT); Sara TRAVAGLIONE, Rome (IT); Pietro ALANO, Rome (IT)

(73) Assignee: ISTITUTO SUPERIORE DI SANITA, Rome (IT)

(21) Appl. No.: 16/319,180

(22) PCT Filed: Jul. 27, 2017
(86) PCT No.: PCT/IB2017/095001

§ 371 Date: Jan. 18, 2019

(30) Foreign Application Priority Data
Jul. 21, 2016 (IT) 102016000076743

(51) Int. Cl. A61K 38/43; A61K 9/00; A61P 33/06

(52) U.S. Cl. CPC A61K 38/43; A61P 33/06; A61K 9/0019; A61K 9/0053

(57) ABSTRACT

The present application relates to the use of bacterial CNF (Cytotoxic Necrotizing Factor) and/or DNT (dermonecrotic toxin) as active ingredients or active ingredient for the prevention and/or the treatment of malaria caused by Plasmodium falciparum, pharmaceutical compositions comprising said active ingredient(s) for said use and a method of prevention and/or treatment of malaria, comprising the administration of bacterial CNF (Cytotoxic Necrotizing Factor) and/or DNT (dermonecrotic toxin) or of a composition comprising it/them to a patient in need thereof.

The present application relates to the use of bacterial CNF (Cytotoxic Necrotizing Factor) and/or DNT (dermonecrotic toxin) as active ingredients or active ingredient for the prevention and/or the treatment of malaria caused by Plasmodium falciparum, pharmaceutical compositions comprising said active ingredient(s) for said use and a method of prevention and/or treatment of malaria comprising the administration of bacterial CNF (Cytotoxic Necrotizing Factor) and/or DNT (dermonecrotic toxin) or of a composition comprising it/them to a patient in need thereof.

### PRIOR ART

Malaria from Plasmodium falciparum is one of the leading causes of disease, neurodisability and death in tropical countries (WHO Malaria Report 2015 doi:ISBN 978 92 4 1564403). In these countries, over 200 million clinical cases of malaria occur every year, and 1% of symptomatic infections can degenerate in severe disease. A severe disease can manifest itself in the form of anemia, hypoglycaemia, metabolic acidosis, repeated seizures, coma or multiple organ dysfunction syndrome. The most severe neurological manifestation of severe malaria is cerebral malaria (CM), causing over one million deaths every year (Snow R W, Guerra C A, Noor A M, Myint H Y, Hay S I. The global distribution of clinical episodes of Plasmodium falciparum malaria. Nature. 2005 Mar. 10; 434(7030):214-7). The clinical characteristic of CM is loss of consciousness, and in the most severe cases coma, caused by cerebral microvessel occlusion due to the thickening of parasite-infested red blood cells (erythrocytes) (pRBCs) adhering to the endothelium. Parasited erythrocytes adhere to the endothelial lining (cytoadherence), with parasite-derived proteins exposed on the erythrocyte surface. The activation of parasite metabolism inside the erythrocyte entails a deep alteration of the host cell metabolism itself and specific structural modifications; in the case of P. falciparum, on the external surface of the erythrocyte, molecules are expressed which mediate adhesion processes of infected erythrocytes to the endothelial cells of cerebral microvessels. Said process, known as ‘sequestration’, constitutes one of the fundamental pathogenetic mechanisms of CM from P. falciparum (Newbold C, Craig A, Kyes S, Rowe A, Fernandez-Reyes D, Fagan T. Cytoadherence, pathogenesis and the infected red cell surface in Plasmodium falciparum. Int J Parasitol. 1999 June; 29(6):927-37). This cytoadherence entails a remarkable adaptive value for the parasite, since, by blocking (sequestering) the infected erythrocyte in deep circulation, it drastically reduces its elimination by the reticuloendothelial system. The molecular mechanisms underlying the ‘sequestration’ phenomenon involve endothelial receptors (CD36, ICAM-1) and parasite proteins, mainly expressed at cytoplasmic extroflections of the infected erythrocyte, referred to as ‘knobs’. In particular, the role of Plasmodium falciparum erythrocyte membrane protein 1 (PfEMP1) is well-described. This protein, encoded by a large family of genes called var, involved in clonal antigenic variability, has a central role in the pathogenesis of severe malaria from P. falciparum and can cause local ischemia.

Little is known about cytoadherence-triggered cell signaling: the adhesion molecules are known to have signal transduction properties that can trigger changes in endothelial cells, such as a remodeling of cytoskeleton and cellular junctions (Etienne-Manneville S, Manneville J B, Adamson P, Wilbourn B, Greenwood J, Couraud P O. ICAM-1-coupled cytoskeletal rearrangements and transendothelial lymphocyte migration involve intracellular calcium signaling in brain endothelial cell lines. J Immunol. 2000 Sep. 15; 165(6):3375-83). The Rho family of monomeric GTPases (comprising the Rho, Rac and Cdc42 subfamilies) plays a central role in the transmission of the various receptors, among which ICAM-1, the vascular cell adhesion molecule (VCAM)-1, and the selectins involved in cytoadherence signaling. Rho GTPases act as molecular switches in the intracellular signaling pathways (active when bound to GTP and inactive when in the GDP-bound form) and represent the first mediators between the above-mentioned receptors and the effectors downstream. In particular, it has been previously demonstrated that adhesion of infected erythrocytes to endothelial cells directly triggers activation of Rho-dependent signaling pathways (Taoufiq Z, Gay F, Balvanyos J, Ciceron L, Tefit M, Lechat P, Mazier D. Rho kinase inhibition in severe malaria: thwarting parasite-induced collateral damage to endothelia. J Infect Dis. 2008 Apr. 1; 197(7):1062-73).

In this context, Escherichia coli Cytotoxic Necrotizing Factor Type 1—(CNF1) a protein toxin that activates the Rho GTPase family (Flatau G, Lemichez E, Gauthier M, Chardin P, Paris S, Fiorentini C, Boquet P. Toxin-induced activation of the G protein p21 Rho by deamidation of glutamine. Nature. 1997 Jun. 12; 387(6634):729-33; Schmidt G, Sehr P, Wilm M, Selzer J, Mann M, Aktories K. Gin 63 of Rho is deamidated by Escherichia coli cytotoxic necrotizing factor-1. Nature. 1997 Jun. 12; 387(6634):725-9; Lerm M, Selzer J, Hoffmeyer A, Rapp U R, Aktories K, Schmidt G. Deamidation of Cdc42 and Rac by Escherichia coli cytotoxic necrotizing factor 1: activation of c-Jun N-terminal kinase in HeLa cells. Infect Immun. 1999 February; 67(2):496-503) and remodels the cell cytoskeleton, can represent a new strategy for thwarting parasite cytoadherence. In fact, by acting on the cytoskeleton, CNF1 protects epithelial cells from apoptosis, promotes macropinocytosis, strengthens natural killer (NK) cell activity and improves astrocytes' ability to sustain neuronal growth and differentiation. It is also interesting to note that CNF1 down-regulates ICAM-1, both in NK/Target cells (Malorni W, Quaranta M G, Straface E, Falzano L, Fabbri A, Viora M, Fiorentini C. The Rac-activating toxin cytotoxic necrotizing factor 1 oversees NK cell-mediated activity by regulating the actin/microtubule interplay. J Immunol. 2003 Oct. 15; 171(8):4195-202) and in epithelial cells (Fiorentini C, Matarrese P, Straface E, Falzano L, Donelli G, Boquet P, Malorni W. Rho-dependent cell spreading activated by E. coli cytotoxic necrotizing factor 1 hinders apoptosis in epithelial cells. Cell Death Differ. 1998 November; 5(11):921-9).

Proteins highly homologous to E. coli CNF1, at least in the catalytic portion, are known in the literature; such proteins are, e.g., E. coli CNF2 and CNF3, Yersinia spp. CNFY or CNF1, and Bordetella spp. DNT (dermonecrotic toxin).

These proteins, as already mentioned, are strongly homologous to CNF1 (as also reported in Medline), above all in the catalytic, and therefore pharmacologically active part, thereof.

To date, artemisinin-based ‘lifesaving’ treatments for CM have mainly related to the intravenous administration of ACT (artemisinin-based combination therapy), (The World Malaria Report, WHO 2015). However, even though these drugs effectively eliminate parasites from blood, 15%-20% of patients die, and the others have severe permanent neurological consequences (Dondorp A, Nosten F, Stepniewska K, Day N, White N; South East Asian Quinine Artesunate Malaria Trial (SEAQUAMAT) group. Artesunate versus quinine for treatment of severe falciparum malaria: a randomised trial. Lancet. 2005 Aug. 27-Sep. 2; 366(9487):717-25; Idro R, Jenkins N E, Newton C R. Pathogenesis, clinical features, and neurological outcome of cerebral malaria. Lancet Neurol. 2005 December; 4(12):827-40). Current treatments for severe malaria from P. falciparum, mainly based on a direct anti-plasmodial strategy, do not seem sufficient, and other complementary approaches are needed to improve the clinical picture of the disease. In this context, the Rho-kinase pathway, which is involved in cytoadherence from P. falciparum (Taoufiq Z, Gay F, Balvanyos J, Ciceron L, Tefit M, Lechat P, Mazier D. Rho kinase inhibition in severe malaria: thwarting parasite-induced collateral damage to endothelia. J Infect Dis. 2008 Apr. 1; 197(7):1062-73), seems a promising pharmacological target for an effective therapeutic approach against malaria. In fact, previous studies show that endothelial cell pre-treatment with Fasudil, a known Rho kinase inhibitor, has protective effects on the endothelium and is able to reduce ICAM-1 adhesion molecule expression (Patent Application CA 2659712 A1-Rho/rock/p13/akt kinase inhibitors for the treatment of diseases associated with protozoan parasites), whereas a pre-treatment with Atorvastatin, a statin which promotes both Rho-kinase inhibition and cell survival by Akt activation, strongly protects the endothelium from apoptosis and is also able to prevent P. falciparum cytoadherence (Taoufiq Z, Pino P, N'dilimabaka N, Arrouss I, Assi S, Soubrier F, Rebollo A, Mazier D. Atorvastatin prevents Plasmodium falciparum cytoadherence and endothelial damage. Malar J. 2011 Feb. 28; 10:52; Taoufiq Z, Gay F, Balvanyos J, Ciceron L, Tefit M, Lechat P, Mazier D. Rho kinase inhibition in severe malaria: thwarting parasite-induced collateral damage to endothelia. J Infect Dis. 2008 Apr. 1; 197(7):1062-73; Waknine-Grinberg J H, Hunt N, Bentura-Marciano A, McQuillan J A, Chan H W, Chan W C, Barenholz Y, Haynes R K, Golenser J. Artemisone effective against murine cerebral malaria. Malar J. 2010 Aug. 9; 9:227). However, these drugs are unable to detach parasites from endothelial cells and to significantly reduce the overall number of parasites present in the body.

Despite decades of research, to date no effective therapeutic approach to severe malaria exists, above all due to its nature of multifactorial syndrome. The main cause of the pathology is due to the process of sequestration of parasite-infected erythrocytes inside capillaries of the encephalon, mediated by the ability to cytoadhere to the vascular endothelium by a ligand-receptor interaction. Said interaction is moreover accountable for the activation of host cell inflammatory processes and the increase of vascular endothelium permeability. At present, OMS suggests treatment with intravenous artesunate, although it cannot prevent all fatal outcomes. Clinical studies based on erythropoietin and/or nitrogen monoxide inhalation are under way.

### SUMMARY OF THE INVENTION

The Authors of the present invention have demonstrated the surprising and unexpected effectiveness of bacterial CNF and/or of proteins homologous thereto, in preventing cytoadherence of infected erythrocytes to endothelial cells, by a downregulation of the expression of the main receptors of the host cell used by the parasite to cytoadhere. Totally new, and even more significant, has proved the CNF1 ability to foster the detachment of infected erythrocytes previously adhered to the endothelium, making the parasite subject to clearance by the spleen.

The latter aspect represents an innovative approach and, to date, the only one with potential to stop and/or slow down processes triggered by adhesion of the infected erythrocyte to the vascular endothelium. The action of inhibiting and reversing the infected erythrocyte ability to adhere by the bacterial CNF and/or proteins homologous thereto represents, to date, an innovative approach in the treatment of severe malaria, with the further advantage of being less subject to the onset of drug resistance phenomena.

Therefore, the invention consists in the use of the purified bacterial protein CNF and/or of proteins homologous thereto as a therapeutic tool against infection from Plasmodium falciparum, one of Plasmodium species causing malaria in humans. The systemic (oral, intravenous) administration of a medicinal composition containing the purified bacterial protein CNF and/or of proteins homologous thereto, could thwart the effects of cerebral malaria and foster parasite elimination by the body. The administration could optionally be carried out in conjunction with additional antimalarial drugs.

By now, it is well-known that an adequate adherence to antimalarial therapies is of paramount importance to improve the results of the treatment, abating the cases of drug-resistant malaria and increasing disease control. In fact, lack of adherence to therapy jeopardizes drug effectiveness itself, above all in malaria-endemic countries: drug-resistant malaria cases could increase and, accordingly, disease management could be even more difficult than it already is (Banek K, Lalani M, Staedke S G, Chandramohan D. Adherence to artemisinin-based combination therapy for the treatment of malaria: a systematic review of the evidence. Malar J. 2014 Jan. 6; 13:7). Specifically, many studies have reported a scarce adherence to the entire cycle of Artemisinin-based combination therapy (ACT), due to the high number of tablets, or because the patient perceives an improvement before the prescribed dose has ended and therefore autonomously decides to interrupt therapy (Onyango E O, Ayodo G, Watsierah C A, Were T, Okumu W, Anyona S B, Raballah E, Okoth J M, Gumo S, Orinda G O, Ouma C. Factors associated with non-adherence to Artemisinin-based combination therapy (ACT) to malaria in a rural population from holoendemic region of western Kenya. BMC Infect Dis. 2012 Jun. 24; 12:143). OMS recommendations on treatment with Primaquine envisage taking the drug for 14-21 days; unfortunately, not all patients adhere to this treatment (Data control and elimination of Plasmodium vivax malaria: a technical brief, WHO 2015), prematurely interrupting the taking of the therapy. The fact that the bacterial CNF and/or proteins homologous thereto be effective after a single administration would therefore eliminate the problem of adherence to therapy, resulting in an effective disease control, with relevant health and economic consequences.

### DETAILED DESCRIPTION OF THE FIGURES

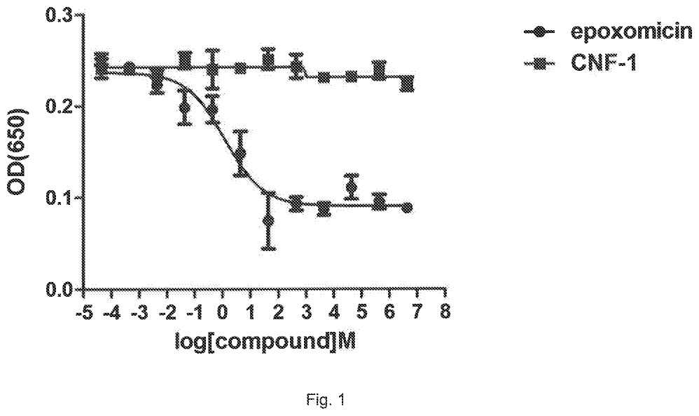

FIG. 1. Dose-response curve for the assessment of CNF1 effect on the parasite, with incubation times of up to 48 h, using the LDH metabolic assay.

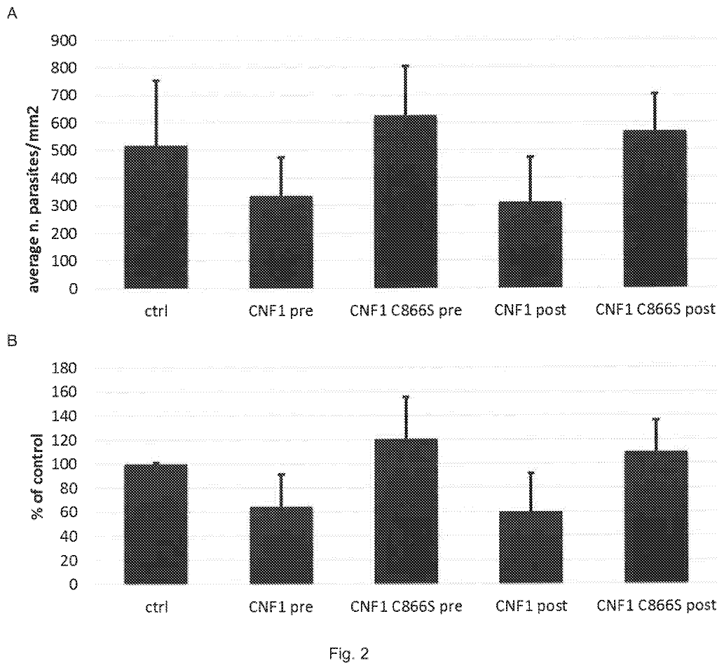

FIG. 2. CNF1 effect on endothelial cytoadherence of P. falciparum. Data expressed as (A) mean number of adhered parasites/mm², and as (B) percentage on the control (n=3).

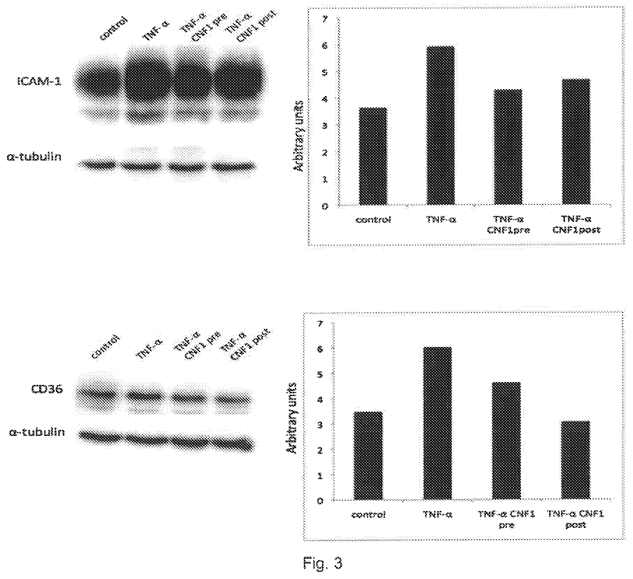

FIG. 3. CNF1 thwarts TNF-α-induced expression increase of endothelial receptors ICAM-1 and CD36. Cells were treated with CNF1 both overnight, before the adhesion process (CNF1 pre) or subsequently to RBCs adhesion assay, for 2 hours (CNF1 post). Cells were lysed and processed for Western blot analysis. ICAM-1 and CD36 amounts were normalized to α-tubulin levels (histograms).

### GLOSSARY

Cytoadherence: occurs when P. falciparum-infected erythrocytes adhere to the endothelium of blood vessels in humans.

Cell cytoskeleton: a large network of interconnected filaments and tubules which extend throughout the cell.

Clearance: in this context, denotes host body ability to eliminate parasites present in the blood via the filtering action of the spleen.

CNF1: acronym for Cytotoxic Necrotizing Factor Type 1, a bacterial protein produced by some pathogenic Escherichia coli strains. CNF1 sequence is known in the literature and reported in public data banks.

CNF2: acronym for Cytotoxic Necrotizing Factor Type 2, a bacterial protein produced by some pathogenic Escherichia coli strains. CNF2 sequence is known in the literature and reported in public data banks.

CNF3: acronym for Cytotoxic Necrotizing Factor Type 3, a bacterial protein produced by some pathogenic Escherichia coli strains. CNF3 sequence is known in the literature and reported in public data banks.

CNFY or CNF1: acronym for Cytotoxic Necrotizing Factor Type Y, a bacterial protein produced by some pathogenic Yersinia pseudotuberculosis strains. CNFY or CNF1 sequence is known in the literature and reported in public data banks.

DNT: acronym for dermonecrotic toxin, a bacterial protein produced by some pathogenic Bordetella spp. strains, in particular by B. pertussis, B. bronchiseptica, B. avium, B. parapertussis.

DNT sequence is known in the literature and reported in public data banks.

ICAM-1 and CD36: the acronyms of, respectively: Intercellular Adhesion Molecule 1 and Cluster of Differentiation 36, cell adhesion molecules involved in cytoadherence.

Synaptic plasticity: is the ability of the nervous system to modify the amount of connections (synapses) among neurons. pRBCs: erythrocytes infected by P. falciparum.

RHO GTPases: a family of small G molecules which act on the actin cytoskeleton of cells, regulating a vast number of cell functions.

### DETAILED DESCRIPTION OF THE INVENTION

As previously mentioned, in the summary of the present invention, the Authors of the invention have surprisingly discovered that the bacterial protein selected from CNF and/or DNT and/or fragments thereof or mixtures thereof that retain the active catalytic portion can be advantageously used in the prevention and/or treatment of malaria caused by Plasmodium falciparum.

In fact, the Authors of the invention have demonstrated that the bacterial proteins reported above (CNF and/or DNT), and as defined in the description and in the claims, prevent the adhesion of erythrocytes infected by Plasmodium falciparum to the endothelium and, above all, induce detachment of infected erythrocytes. Without wishing to be bound by theory, the Authors hypothesize that the effect of the above-indicated proteins on the binding of erythrocytes infected by P. falciparum to endothelial cells depends on a coordinated modulation of processes linked to cytoskeleton motility and plasticity. The remodeling of the cytoskeleton of infected erythrocytes, induced by treatment with the above-defined proteins, might explain the loss of parasite ability to cytoadhere and might promote their detachment from the vascular endothelium, with the entailed “elimination” of the infected erythrocytes from peripheral circulation. The authors have also surprisingly discovered and demonstrated the effectiveness of the bacterial proteins reported above (CNF and/or DNT), and as defined in the description and in the claims, not only in preventing the cytoadherence of infected erythrocytes to endothelial cells, by a downregulation of the expression of the main receptors of the host cell used by the parasite to cytoadhere, but also in fostering the detachment of infected erythrocytes previously adhered to the endothelium, making the parasite subject to clearance by the spleen.

This latter aspect represents an innovative approach and, to date, the only one with the potential to stop and/or slow down the processes triggered by the adhesion of the infected erythrocyte to the vascular endothelium. The action of inhibition and reversion of the infected erythrocyte ability to adhere by the bacterial proteins reported above (CNF and/or DNT) and as defined in the description and in the claims, would represent, to date, an innovative approach in the treatment of severe malaria with the further advantage of being less subject to the onset of drug resistance phenomena.

Therefore, object of the present invention is a bacterial protein selected from CNF and/or DNT and/or fragments thereof that retain the active catalytic portion, or mixtures thereof, for use in the prevention and/or treatment of malaria caused by Plasmodium falciparum.

By ‘fragments that retain the active catalytic portion’ there are meant fragments of the proteins as defined in the present description and in the claims, as long as such fragments retain the catalytic portion and said catalytic portion has the activity of the wild-type protein.

According to the present description, the protein for use in the prevention and/or treatment of malaria caused by Plasmodium falciparum can be selected from Escherichia coli CNF1, CNF2, CNF3; Yersinia spp. (e.g., Yersinia tuberculosis or pseudotuberculosis) CNFY or CNF1; Erwinia spp. CNF; Bordetella spp. DNT or mixtures thereof. Furthermore, for the same preventive and/or therapeutic aim, one or more fragments of the above-described proteins can be used, as long as such fragments retain the catalytic portion and said catalytic portion has the activity of the wild-type protein.

According to one embodiment of the present description, said Bordetella spp. DNT is selected from B. pertussis DNT and/or B. bronchiseptica B. DNT and/or B. avium DNT and/or B. parapertussis DNT or from mixtures thereof.

In particular, according to the present invention bacterial proteins homologous to E. coli CNF1 protein and having the same function can be used.

According to the present invention, the CNF1 (Cytotoxic Necrotizing Factor Type 1) protein corresponds to the protein produced by some pathogenic Escherichia coli strains. CNF1 sequence is known in the literature and reported in public data banks; no further information is needed for a technician in the field to easily carry out the invention.

According to the present invention, the CNF2 (Cytotoxic Necrotizing Factor Type 2) protein corresponds to the protein produced by some pathogenic Escherichia coli strains. CNF2 sequence is known in the literature and reported in public data banks; no further information is needed for a technician in the field to easily carry out the invention.

According to the present invention, the CNF3 (Cytotoxic Necrotizing Factor Type 3) protein corresponds to the protein produced by some pathogenic Escherichia coli strains. CNF3 sequence is known in the literature and reported in public data banks; no further information is needed for a technician in the field to easily carry out the invention.

According to the present invention, the CNFY (Cytotoxic Necrotizing Factor Type Y) or CNF1 protein corresponds to the protein produced by Yersinia pseudotuberculosis bacteria. CNFY or CNF1 sequence is known in the literature and reported in public data banks; no further information is needed for a technician in the field to easily carry out the invention.

According to the present invention, the DNT (dermonecrotic toxin) protein corresponds to the protein produced by bacteria belonging to Bordetella spp., whose sequence is known in the literature. In the preferred embodiments of the present invention, the protein could be, e.g., a DNT from B. pertussis, B. bronchiseptica, B. avium, B. parapertussis or mixtures thereof. The sequences of the various DNTs are known in the literature and reported in public data banks; no further information is needed for a technician in the field to easily carry out the invention.

A non-limiting example of proteins suitable for the carrying out of the present invention is reported in table 1 below, where entry name and organism of CNF or DNT proteins are indicated as available to the public in UNIPROT databank on 07/21/2016.

TABLE 1

| Entry name | Organism |
|---|---|
| W6AYD9_9GAMM | Moritella viscosa |
| Q47106_ECOLX | Escherichia coli |
| Q7VTS2_BORPE | Bordetella pertussis (strain Tohama I/ATCC BAA-589/NCTC 13251) |
| Q1R2U0_ECOUT | Escherichia coli (strain UTI89/UPEC) |
| Q2KUX5_BORA1 | Bordetella avium (strain 197N) |
| Q7W4W3_BORPA | Bordetella parapertussis (strain 12822/ATCC BAA-587/NCTC 13253) |
| A0A0C6PDA9_BORBO | Bordetella bronchiseptica 253 |
| A0A0H3LPZ7_BORBR | Bordetella bronchiseptica (strain ATCC BAA-588/NCTC 13252/RB50) |
| | (Alcaligenes bronchisepticus) |
| Q47107_ECOLX | Escherichia coli |
| B2VHY9_ERWT9 | Erwinia tasmaniensis (strain DSM 17950/CIP 109463/Et1/99) |
| Q8KTM3_ECOLX | Escherichia coli |
| E3DDI9_ERWSE | Erwinia sp. (strain Ejp617) |
| Q1M2S9_ECOLX | Escherichia coli |
| Q8KTM4_ECOLX | Escherichia coli |
| Q46962_ECOLX | Escherichia coli |
| A0A125XP14_ECOLX | Escherichia coli 2-460-02_S1_C2 |
| A0A168S6Z2_ECOLX | Escherichia coli |
| Q45336_BORPT | Bordetella pertussis |
| A0A0T7CRY6_BORP1 | Bordetella pertussis (strain ATCC 9797/DSM 5571/NCTC 10739/18323) |
| Q45044_BORBO | Bordetella bronchiseptica (Alcaligenes bronchisepticus) |
| C5ZZQ2_ECOLX | Escherichia coli Vir68 |
| A0A0K5ZZ47_ECOLX | Escherichia coli |
| A0A0A0XE42_BORPT | Bordetella pertussis B1920 |
| Q9EYH7_YERPU | Yersinia pseudotuberculosis |
| Q0E668_ECOLX | Escherichia coli |
| A0A0U0VI01_BORPT | Bordetella pertussis |
| A0A0N9JNY6_YERPU | Yersinia pseudotuberculosis |
| A0A0H3B5B9_YERPY | Yersinia pseudotuberculosis serotype O:3 (strain YPIII) |
| D0Z517_PHODD | Photobacterium damselae subsp. damselae CIP 102761 |
| A0A063UW49_BORBO | Bordetella bronchiseptica OSU553 |
| Q9S5D5_BORBO | Bordetella bronchiseptica (Alcaligenes bronchisepticus) |
| I0VKY3_ECOLX | Escherichia coli W26 |
| A0A0L1C6V4_ECOLX | Escherichia coli |
| A0A026RKM1_ECOLX | Escherichia coli O119:H4 str. 03-3458 |
| A0A0W3D0A0_ESCFE | Escherichia fergusonii |
| A0A0N2IV24_BORPT | Bordetella pertussis H921 |

According to the invention, any one of these proteins or mixtures thereof, as well as fragments thereof (mixed to each other or with one or more of the above-described proteins) that retain the active catalytic portion, could be utilized for use in the prevention and/or treatment of malaria caused by Plasmodium falciparum.

The protein or the active fragments of the protein as described herein could be directly, or even recombinantly produced by the above-indicated bacteria, and could be purified by standard methods commonly used in the field.

As described above and shown in the experimental section, the protein or the mixture of proteins or fragments thereof as defined in the present description and in the claims have surprisingly shown not only the ability to prevent cytoadherence of Plasmodium falciparum-infected erythrocytes to endothelial cells, but also that of fostering the detachment of Plasmodium falciparum-infected erythrocytes previously adhered to the endothelium, thereby providing not only a preventive effect, but also a truly therapeutic effect that enables a clearance of the parasite by the spleen. The effect of the proteins or of fragments thereof, as defined herein and as defined in the claims, therefore enable to carry out over time a real eradication of the parasite from the patient's blood.

According to the present description, the protein or mixture of proteins as defined in the description and in the claims, could be used for a therapeutic treatment envisaging the administration thereof in one or more doses to a patient in need thereof.

The patient could therefore be an individual who potentially has to expose him/herself to the parasite, or an individual already affected by malaria.

The protein or the mixture of proteins or fragments thereof for use in the prevention and/or treatment of malaria caused by Plasmodium falciparum could be administered by systemic administration, like e.g. by oral, intravenous, transmucosal, percutaneous, rectal, sublingual route, by inhalation or by aerosol.

The protein or mixture of proteins or fragments thereof could be administered in one or more doses; therefore, also an administration carried out in a single dose is envisaged.

Object of the invention is also a pharmaceutical composition comprising a bacterial protein selected from CNF and/or DNT and/or fragments thereof that retain the active catalytic portion, or mixtures thereof, and at least one pharmaceutically acceptable carrier for use in the prevention and/or treatment of malaria caused by Plasmodium falciparum.

All of the above-provided definitions and descriptions of the proteins and of the active fragments thereof also apply to the embodiments of the composition of the invention.

Therefore, object of the invention is a pharmaceutical composition for use in the prevention and/or treatment of malaria caused by Plasmodium falciparum, wherein said protein can be selected from Escherichia coli CNF1, CNF2, CNF3; Yersinia tuberculosis CNFY or CNF1; Bordetella spp. DNT, fragments thereof that retain the active catalytic portion, or mixtures thereof.

Moreover, wherein said Bordetella spp. DNT could be selected from B. pertussis DNT and/or B. bronchiseptica DNT and/or B. avium DNT and/or B. parapertussis DNT, fragments thereof that retain the active catalytic portion, or mixtures thereof.

A person skilled in pharmaceutical compositions could select without particular difficulties the carriers and any excipients, preservatives, flavorings and further additives commonly used in the practice of pharmaceutical preparations.

According to the invention, the pharmaceutical composition could therefore be made in the form of suspension for injection, for oral use of for inhalation; of an emulsion, of a tablet, of a capsule, of a hard or soft gelatin, of a suppository, of an enema, of a syrup, of an elixir and the like.

The composition could further be formulated in single dose units, in dosable aliquots or in single-dose formulations.

The prevention and/or treatment could therefore be carried out by administration thereof in one or more doses to a patient in need thereof.

According to one embodiment, the administration of the pharmaceutical composition of the invention could be carried out by systemic administration, like, e.g. by oral, intravenous, transmucosal, percutaneous, rectal, sublingual route, by inhalation or by aerosol and the like.

The following section serves to show some experimental data obtained by the Authors of the invention, as well as to jointly exemplify, in a non-limiting way, embodiments of the invention.

### EXAMPLES AND EXPERIMENTAL DATA

Treatment with CNF1 does not Influence Parasite Vitality:

The hypothetical toxic activity of CNF1 towards the parasite was tested beforehand by using LDH metabolic assay, in dose-response experiments with incubation times of up to 48 h. The results showed that CNF1 has no significant effect on parasite growth and/or multiplication ability in the range of concentrations used (FIG. 1).

CNF1 reduces infected erythrocytes cytoadherence to endothelial cells: The Authors studied the effect of CNF1 on the adhesion of clone parasite ITG, able to efficiently bind through ICAM-1 and CD36 (Ockenhouse C F, Ho M, Tandon N N, Van Seventer G A, Shaw S, White N J, Jamieson G A, Chulay J D, Webster H K. Molecular basis of sequestration in severe and uncomplicated Plasmodium falciparum malaria: differential adhesion of infected erythrocytes to CD36 and ICAM-1. J Infect Dis. 1991 July; 164(1):163) to endothelial cell line HBMEC-60, in the presence or absence of an activation phase at 12 h with 100 U/ml rTNF-alpha. HBMEC-60 monolayer cells were: 1) preincubated overnight with CNF1 before being exposed to pRBCs; 2) incubated with CNF1 for 2 h after pRBCs binding.

The data showed that parasite cytoadherence decreased by 48%±10 compared to controls not pretreated with CNF1 (10⁻¹⁰ M). It is interesting to note that 2 h of incubation with CNF1 after pRBCs binding with the endothelium led to a 40% decrease of the pRBCs bound to the endothelial monolayer compared to the control. This effect on cytoadherence was not observed by using the CNF1 C866S recombinant, which does not have the ability to activate Rho GTPases. Therefore, CNF1 is not only able to prevent adhesion of the P. falciparum-infected erythrocyte to endothelial cells, but is also able to “detach” the parasite-infected erythrocyte from endothelial cells. Moreover, the non-observance of this effect utilizing CNF1 C866S strongly suggests that Rho GTPases activity is crucial in this process (FIG. 2).

CNF1 Inhibits the Expression of ICAM-1 and CD36:

pRBCs cytoadherence to blood microcirculation endothelia involves PfEMP-1 and receptors such as CD36 and ICAM-1 (Almelli T, Ndam N T, Ezimegnon S, Alao M J, Ahouansou C, Sagbo G, Amoussou A, Deloron P, Tahar R. Cytoadherence phenotype of Plasmodium falciparum-infected erythrocytes is associated with specific pfemp-1 expression in parasites from children with cerebral malaria. Malar J. 2014 Aug. 25; 13:333). Therefore, to explain the mechanism with which CNF1 was able to “detach” infected red cells from endothelial cells, the Authors analyzed the expression of these receptors via Western blot techniques under two different experimental conditions: (i) on cells pretreated with CNF1 overnight prior to the adhesion process (ii) on cells treated 2 h with the toxin after iRBCs adhesion. The results in FIG. 3 clearly show CNF1 ability to thwart the increase of induced TNF-α of a CD36 and ICAM-1 under both experimental conditions (FIG. 3).

CNF1 Preparation

CNF1 was obtained from the 392 ISS strain (provided by V. Falbo, Rome, Italy) and purified as previously described (Falzano L, Fiorentini C, Donelli G, Michel E, Kocks C, Cossart P, Cabanié L, Oswald E, Boquet P. Induction of phagocytic behaviour in human epithelial cells by Escherichia coli cytotoxic necrotizing factor type 1. Mol Microbiol. 1993 September; 9(6):1247-54). The recombinant protein CNF1 C866S (mCNF1), in which the enzymatic activity on Rho GTPases was abrogated by change of cystein with serine at position 866 (Schmidt G, Selzer J, Lerm M, Aktories K. The Rho-deamidating cytotoxic necrotizing factor 1 from Escherichia coli possesses transglutaminase activity. Cysteine 866 and histidine 881 are essential for enzyme activity. J Biol Chem. 1998 May 29; 273(22):13669-74), was used as a control. The plasmid coding for CNF1 C866S, purified as previosly described by Falzano et al. (1993), was kindly provided by E. Lemichez (U627 INSERM, Nice, France).

Parasite Cultures

The ITG line of P. falciparum (Ockenhouse et al. 1991) was cultivated in 0+ human erythrocytes at 5% haematocrit (HTC), using a 5% CO₂, 2% O₂, 93% N₂ atmosphere. As culture medium, RPMI 1640 (Gibco) additioned with 25 mM Hepes, hypoxanthine 50 pg/mL, 0.25 mM NaHCO₃, 50 mg/ml gentamicin sulfate, and 10% heat-inactivated 0+ human serum was used. For synchronization of the desired stages of the parasite, 5-8% parasitaemia and 5% haematocrit cultures were centrifuged at 2000 rpm for 10 minutes, resuspended in incomplete medium (RPMI 1640 additioned with 6 mM glucose, pH 7.2) and laid on a 60% Percoll cushion (Sigma). Before the assay, the samples were washed twice in incomplete medium and resuspended in 3% parasitaemia and 1% haematocrit.

In Vitro Drug Susceptibility Assay

CNF1 was diluted in a medium complete with the parasites, and serial dilutions were carried out in a 96-well plate (Euroclone), to a final volume of 100 μl/well. Trophozoites/schizonts deriving from an asynchronous parasite culture, with a 1-1.5% parasitaemia, were distributed in the plate (100 μl/well, final haematocrit 1%) and incubated 48 hours at 37° C. Epoxomicin (Sigma E3652) was used as a control for asexual parasites. Experiments were conducted three times in duplicate. Parasite growth was determined at the spectrophotometer, by measuring the activity of the parasite lactate dehydrogenase (pLDH), following the protocol described in Makler M T, Ries J M, Williams J A, Bancroft J E, Piper R C, Gibbins B L, Hinrichs D J. Parasite lactate dehydrogenase as an assay for Plasmodium falciparum drug sensitivity. Am J Trop Med Hyg. 1993 June; 48(6):739-41. In short, CNF1-treated cultures were resuspended, and 20 μl/well were transferred into a plate containing 100 μl of malstat reagent [0.11% v/v Triton-100; 115.7 mM 123 lithium L-lactate; 30.27 mM Tris; 0.62 mM 3-acetylpyridine adenine dinucleotide (APAD; Sigma-124 Aldrich); brought to pH 9 with 1 M HCl] and 25 μl di PES/NBT (1.96 mM nitro blue tetrazolium 125 chloride; 0.24 mM phenazine ethosulphate). The plate was read at a 650 nm wavelength, using a Synergy4 (BioTek) microplate reader, and the results were expressed as inhibiting concentration (IC50).

Endothelial Cells

Immortalized human bone marrow-derived endothelial cells (HBMEC-60) were kindly provided by Dr. E Van der Schoot (CLB, Sanquin Blood Supply Foundation, The Netherlands). HBMEC-60 were grown in 1% gelatin-coated flasks, using a culture medium specific for endothelial cells (EBM-2 BulletKit, Lonza).

Static Binding Assay

Endothelial cells were seeded onto 1% gelatin coated 13 mm 0 coverslips (Nalgene, Nunc). Once confluent, the cells were incubated overnight at 37° C. with 1 ng/ml rTNF-alpha (Invitrogen UK). Then, they were washed with DMEM and incubated with 0.5 ml of parasite suspension (3% parasitaemia and 1% haematocrit) for 2 hours at 37° C. After two washes in RPMI 1640, cells were treated with CNF1 and mCNF1 for 2 hours. Coverslips were then washed again with RPMI 1640 twice for 30 minutes, to remove parasites unbound to cells. Finally, cells were fixed with 1% glutaraldehyde and stained with 5% Giemsa for 30 minutes. Each experimental condition was repeated three times and parasites were counted at 400× magnification. Their number was expressed as number of infected red blood cells per mm².

Protein Extraction and Western Blot

Cells were lysed in Sample Buffer 1× (50 mM Tris-HCl pH 6.8, 2% SDS, 10% glycerol, 100 mM DTT) preheated to 100° C. 30 μg of total protein extracts were separated on 8% SDS-PAGE polyacrylamide gel and then electrically transferred onto PVDF filters. After having saturated the free binding sites with TBS-T (20 mM Tris-HCl (pH 7.4), 150 mM NaCl, 0.02% Tween-20) containing 5% skim milk (BIORAD), filters were incubated overnight at 4° C. with the following primary antibodies diluted in TBS-T containing 2% milk: anti-ICAM-1 mouse monoclonal antibody (Santa Cruz Biotechnology, 1:500 dilution), anti-CD36 rabbit polyclonal antibody (Santa Cruz Biotechnology, 1:500 dilution), anti-alpha tubulin mouse monoclonal antibody (Sigma-Aldrich, 1:10000 dilution). After numerous washes in TBS-T, the immune complexes were detected with horseradish peroxidase-conjugated species-specific secondary antiserum (Jackson Laboratory), followed by a reaction based on the chemiluminescence method, ECL (Enhanced Chemiluminescence Detection, Millipore Corporation). Proteins detected by immunoblotting were quantitated by densitometry (ChemiDoc imaging system, BioRad) and normalized to α-tubulin expression levels by means of the Image-Lab (Bio-Rad) program.

1-13. (canceled)

14. A method for the prevention and/or treatment of cerebral malaria caused by Plasmodium falciparum wherein said prevention and/or treatment is carried out by systemic (oral, intravenous) administration in a single dose of a bacterial protein selected from CNF (Cytotoxic Necrotizing Factor) and/or DNT (Dermonecrotic Toxin) and/or fragments thereof that retain the active catalytic portion, or mixtures thereof to a patient in need thereof.

15. The method according to claim 14, wherein said protein is selected from Escherichia coli CNF1, CNF2, CNF3; Yersinia tuberculosis CNFY or CNF1; Bordetella spp. DNT, fragments thereof that retain the active catalytic portion, or mixtures thereof.

16. The method according to claim 15, wherein said Bordetella spp. DNT is selected from B. pertussis DNT and/or B. bronchiseptica DNT and/or B. avium DNT and/or B. parapertussis DNT, fragments thereof that retain the active catalytic portion, or mixtures thereof.

17. The method of claim 16, wherein said protein or mixture administration is a systemic administration by oral, intravenous route.

18. A method for the prevention and/or treatment of cerebral malaria caused by Plasmodium falciparum wherein said prevention and/or treatment is carried out by systemic (oral, intravenous) administration in a single dose of a pharmaceutical composition comprising a bacterial protein selected from CNF (Cytotoxic Necrotizing Factor) and/or DNT (Dermonecrotic Toxin) and/or fragments thereof that retain the active catalytic portion, or mixtures thereof; and at least one pharmaceutically acceptable carrier; to a patient in need thereof.

19. The method according to claim 18, wherein said protein is selected from Escherichia coli CNF1, CNF2, CNF3; Yersinia tuberculosis CNFY or CNF1; Bordetella spp. DNT, fragments thereof that retain the active catalytic portion, or mixtures thereof.

20. The method according to claim 19, wherein said Bordetella spp. DNT is selected from B. pertussis DNT and/or B. bronchiseptica DNT B. and/or B. avium DNT and/or B. parapertussis DNT, fragments thereof that retain the active catalytic portion, or mixtures thereof.

21. The method according to claim 18, wherein said pharmaceutical composition is a composition for intravenous administration is in the form of a suspension for injection.

22. The method according to claim 18, wherein said pharmaceutical composition is a composition for oral administration in the form of an emulsion, a tablet, a capsule, a hard gelatin, a soft gelatin, a syrup or an elixir.
